(19) United States
(12) Patent Application Publication
(10) Pub. No.: US 2006/0160131 A1
(43) Pub. Date: Jul. 20, 2006

(54) Method for the identification of drug targets

(76) Inventors: Joel Vandekerckhove, Loppem (BE); Kris Gevaert, Zele (BE)

Correspondence Address: NIXON & VANDERHYE, PC, 901 NORTH GLEBE ROAD, 11TH FLOOR, ARLINGTON, VA, 22203, US

(21) Appl. No.: 10/527,662

(22) PCT Filed: Sep. 11, 2003
(86) PCT No.: PCT/EP03/50402

§ 371 Date: Mar. 11, 2005

(30) Foreign Application Priority Data
Sep. 12, 2002 (EP) 02078801.4

(51) Int. Cl. G01N 33/53; C12Q 1/37

(52) U.S. Cl. 435/7.1; 435/23

(57) ABSTRACT

The present invention relates to the field of drug development. More specifically the invention provides a method for the identification of drug targets. The method can also be used for analysis of proteomes. The method utilizes in essence a combination of two chromatographic separations of the same type, separated by a step in which the population of the drug-bound targets is altered specifically on the drug in such a way that the chromatographic behaviour of the altered drug-bound targets in the second chromatographic separation differs from the chromatographic behaviour of its unaltered version. The different chromatographic behaviour of the altered drug-bound targets is used for the isolation and subsequent identification of the targets.

### FIELD OF THE INVENTION

The present invention relates to the field of drug development. More specifically the invention provides a method for the identification of drug targets. The method can also be used for analysis of proteomes. The method utilizes in essence a combination of two chromatographic separations of the same type, separated by a step in which the population of the drug-bound targets is altered specifically on the drug in such a way that the chromatographic behaviour of the altered drug-bound targets in the second chromatographic separation differs from the chromatographic behaviour of its unaltered version. The different chromatographic behaviour of the altered drug-bound targets is used for the isolation and subsequent identification of the targets.

### BACKGROUND OF THE INVENTION

Now in the post-genome era, many strategies for the analysis of proteins are currently being developed. Most conventional approaches focus on recording variations in protein level. These approaches are commonly referred to as “proteomics”. In general, proteomics seeks to measure the abundance of broad profiles of proteins from complex biological mixtures. In the most common embodiments, proteomics involves separating the proteins within a sample by two-dimensional SDS-PAGE. Then, the individual protein spot patterns of these gels can be compared to get indications as to the relative abundance of a particular protein in two comparative samples. The approach can even be extended to determine the molecular identity of the individual protein spots by excising the spots and subjecting them to peptide mass fingerprinting. More recently, methods have been described for eliminating the electrophoresis steps and performing proteomics by directly analyzing the complex mixture by mass spectrometry. For example, methods currently described in the art provide chemically reactive compounds that can be reacted with a protein mixture to label many proteins in that mixture in a non-specific, or non-directed, manner providing only a quantitative analysis of proteins (Link et al. (1999) Nat. Biotechnol. 17, 676-682, Gygi et al. (1999) Nat. Biotechnol. 17, 994-999). Such methods teach that there are many chemically reactive amino acid residues within a protein which can be conjugated to chemical probes, whereby the resulting protein complexes can be subsequently quantified to yield an indication of protein abundance. In WO 01/77668 the use of activity-based probes (ABP) is described to screen for target proteins of said ABPs. In this technology the ABPs are coupled with an affinity ligand that serves to detect the drug-target complexes. There is however an urgent need to develop methods that allow a more detailed analysis of a complex mixture of proteins or even of a whole proteome. It is well known that the control (activation or inhibition) of protein activities in a cell is due to changes in the protein structure available to other components in the cell. Conformational changes and movements in hinge regions of proteins expose specific parts of these proteins and allow them to contact compounds such as enzyme substrates, adaptor proteins, and other components such as drugs. Moreover, the activity of drugs is due to the specific interaction with proteins influencing their biological activity. In several cases the protein targets of existing drugs are known: e.g. aspirin reacts with the cyclo oxygenases, penicillin is a pseudo substrate of the peptide glycan amino transferase of Gram+bacteria, etc. Also in some exceptional cases, drugs have been designed and improved based on the 3D-structure of the target protein. In most cases, however components with biological activities have not yet been allocated to their target proteins and hence the targets of most drugs are unknown. The reliable identification of the targets of existing drugs or drugs in development would be extremely valuable for the estimation of the specificity and prediction of side effects of drugs. Furthermore it is known that the inter-individual response to drugs varies considerably. The aim of modern drug development is to generate tailor made drugs that are efficient for individual patient categories. The present invention relates to a solution to the above-cited problems and discloses a method to determine the interaction partners of drugs and also the interaction site in the primary structure of the target protein. The method can be used to estimate a correlation between the disease response to a certain drug with the targets of said drug identified in individual patients or patient groups. Our method is independent of the use of detectable or affinity labels that are coupled to the drugs, as described in WO 01/77668. In addition, the method offers the advantage that the drug targets can be efficiently isolated in a chromatographic step. In addition the site in the primary structure or the protein target on which the drug binds can be efficiently determined with the current invention.

### BRIEF DESCRIPTION OF THE FIGURES

FIG. 1A): Actin was incubated with a target peptide “CP” and cross-linked by transglutaminase. The cross-linked components were digested with endo-Lys-C. The UV-absorption profile of endo-Lys-C peptides separated on a C-18 reversed phase column (run 1) is shown in FIG. 1A. Solvent A is 0.1% TFA, solvent B is 70% acetonitrile in 0.1% TFA-water. The gradient of solvent B is indicated. Eluting peptides are collected in 5 min. wide intervals and dried B). Fraction 6, containing the cross-linked peptide was rerun in the same chromatographic conditions as in run 1 after specific cleavage with factor Xa. The shifted peptide carrying the cross-link is visible in FIG. 1B in front of the bulk of unmodified peptides (in black).

FIG. 2: The cross-linked peptide, shifting in front of fraction 6 (FIG. 1B) was analysed by electrospray ionization mass spectrometry. The differently charged peptide ions are shown and allow the determination of the mass of this cross-linked dipeptide. The analysis was carried out on a Micromass Q-TOF apparatus.

FIG. 3A): A total lysate of Jurkat cells was digested with endo-Lys-C. This peptide mixture was mixed with a similar digest of the actin-CP conjugate. The peptide mixture was separated by reversed-phase chromatography as in FIG. 1A. The first part of the chromatogram was recorded at AUFS 0.1, the second part at AUFS 0.2. The eluate was collected in fractions of 2 min. These fractions were dried and recombined as in Table 1 before being treated with factor Xa.

FIG. 3B) shows the UV traces of the peptides in pool D (see Table 1). The profiles of primary fractions 9, 14 and 19 are shown. 9* is a peak eluting in front of the bulk of peptides. 9** is peptide Ac-F-1-E-G-R derived from excess of CP and cleaved by factor Xa. Note a peak (in dark) eluting in front of fraction 14. All chromatographic conditions were as in the experiment of FIG. 1.

### AIMS AND DETAILED DESCRIPTION OF THE INVENTION

The present invention provides an alternative method for the isolation and identification of drug targets. The method also allows the quantitation of expression levels and/or activities of classes of proteins or/and enzymes or individual proteins or/and enzymes in a global cell lysate background. The method utilizes in essence a combination of two chromatographic separations of the same type, separated by a step in which the population of the drug-bound targets is altered specifically on the drug in such a way that the chromatographic behaviour of the altered drug-bound targets in the second chromatographic separation differs from the chromatographic behaviour of its unaltered version. The different chromatographic behaviour of the altered drug-bound targets is used for the isolation and subsequent identification of the targets.

In one embodiment the invention provides a method to isolate at least one target molecule of a compound comprising a functional group that can be specifically altered, said method comprises the following steps (a) adding said compound to a complex mixture of molecules wherein said compound stably interacts with at least one molecule forming a compound-target complex, (b) separating the resulting complex mixture of molecules and compound-target complexes into fractions via chromatography, (c) chemically, or enzymatically, or chemically and enzymatically altering said compound present on at least one compound-target complex in each fraction, and (d) isolating at least one target molecule that interacts with said compound via chromatography, wherein the chromatography of steps (b) and (d) is performed with the same type of chromatography.

In another embodiment the invention provides a method to isolate at least one target protein of a compound comprising a functional group that can be specifically altered. Said method comprises the following steps (a) adding said compound to a complex mixture of proteins wherein said compound stably interacts with at least one target protein forming a compound-protein complex, (b) separating the resulting complex mixture of proteins and compound-protein complexes into fractions via chromatography, (c) chemically, or enzymatically, or chemically and enzymatically altering said compound present on at least one compound-protein complex in each fraction, and (d) isolating at least one target protein that interacts with said molecule via chromatography, wherein the chromatography of steps (b) and (d) is performed with the same type of chromatography.

In another embodiment the invention provides a method to isolate at least one target peptide of a compound comprising a functional group that can be specifically altered. Said method comprises the following steps (a) adding said compound to a complex mixture of proteins wherein said compound stably interacts with at least one target protein forming a compound-protein complex, (b) cleaving the resulting complex protein mixture and compound-protein complexes into a protein peptide mixture, (c) separating said protein peptide mixture into fractions via chromatography, (d) chemically, or enzymatically, or chemically and enzymatically altering said compound present on at least one compound-peptide complex in each fraction and (e) isolating at least one target peptide that interacts with said compound via chromatography wherein the chromatography of steps (c) and (e) is performed with the same type of chromatography.

In yet another embodiment the invention provides a method to isolate at least one target of a compound comprising a functional group that can be specifically altered wherein said compound is added directly to a protein peptide mixture and wherein said compound stably interacts with at least one target peptide forming a compound-peptide complex.

In yet another embodiment the chromatographic conditions used in the preceding methods are the same or substantially similar.

As used herein, a “protein peptide mixture” is typically a complex mixture of peptides obtained as a result of the cleavage of a sample comprising proteins. Such sample is typically any complex mixture of proteins such as, without limitation, a prokaryotic or eukaryotic cell lysate or any complex mixture of proteins isolated from a cell or a specific organelle fraction, a biopsy, laser-capture dissected cells or any large protein complex such as ribosomes, viruses and the like. It can be expected that when such protein samples are cleaved into peptides that they may contain easily up to 1.000, 5.000, 10.000, 20.000, 30.000, 100.000 or more different peptides. However, in a particular case a “protein peptide mixture” can also originate directly from a body fluid or more generally any solution of biological origin. It is well known that, for example, urine contains, besides proteins, a very complex peptide mixture resulting from proteolytic degradation of proteins in the body of which the peptides are eliminated via the kidneys. Yet another illustration of a protein peptide mixture is the mixture of peptides present in the cerebrospinal fluid.

The term ‘at least one target of a compound’ means that a particular compound stably interacts with one or more target molecules, or a class of molecules. The binding of a compound to the target is specific, meaning that said compound binds to at least one molecule in a complex mixture of molecules and not to other molecules. Usually a compound is a drug, a drug analogue or drug derivative. Preferably said binding causes an inactivation or a partial inactivation of the molecule (e.g. inhibits its activity) and the binding preferably occurs at the active site of the molecule (e.g. of a protein). Since the binding occurs at the active site of a protein the method of the present invention can also be used for the isolation of a specific class of active proteins. Active means that the active site is accessible for the compound whereas inactive proteins of the same class will not be isolated because the active site is not accessible for the compound.

Here an ‘active site’ of a protein refers to the specific area on the surface of a protein (e.g. an enzyme or receptor), to which a compound (e.g. a substrate, a ligand, a drug or a drug analogue or a drug derivative) can bind resulting in a change in the configuration of the protein. With regard to a receptor, due to the conformational change, the protein may become susceptible to phosphorylation or dephosphorylation or other processing. With regard to other proteins the active site will be the site(s) where the substrate and/or cofactor or drug or drug analogue or drug derivative binds or where the substrate and cofactor undergo a catalytic reaction, or where two proteins form a complex, (e.g. two kringle structures bind, sites at which transcription factors bind to other proteins, sites at which proteins bind to specific nucleic acid sequences, etc.).

The ‘compounds’ of the invention are chemical reagents that are poly-functional agents for non-competitive or substantially irreversible binding to a target molecule. ‘Compounds’ comprise small compounds (organic or inorganic), existing drugs, drugs in development, drug leads, drug analogues or drug derivatives. An individual compound, a subset of compounds or the complete set of compounds derived from a library of compounds such as a library established by combinatorial chemistry. In most general terms, the compound consists of (1) a chemical structure determining the specific interaction between said compound and its target molecule (the “S”-part), (2) a chemically reactive group by which the compound and its target can be tightly cross-linked (the “L”-part) and (3) a functional group which can be altered on a specific and controllable manner (the “A”-part). These three properties (“S” for specificity, “L” for cross-linking and “A” for alteration) can be differently distributed over the compound structure.

According to the invention a compound-target complex is chemically, or enzymatically, or chemically and enzymatically altered between the two chromatographic separations. In a preferred embodiment a compound is a drug, a drug analogue or drug derivative. A drug derivative is a drug (for example an existing drug) on which an extra group is attached such as for example an alteration part (“A” part) or a functional group by which the compound and its target can be tightly cross-linked (“L” part). Said “A” group or “A” part is necessary and sufficient for the chemical or enzymatic or chemical and enzymatic alteration between the two chromatographic separations.

In order to distinguish the “S”, “L” and “A” part of a target molecule from the one-letter notation of the amino acids Ser (S), Leu (L) and Ala (A) used in this description of the invention; S, L and A will be used to define their corresponding amino acids, while “S”, “L” and “A”, or “S”-part, “L”-part and “A”-part, or “S”-moiety, “L”-moiety and “A”-moiety will be used to indicate functional entities within the compounds: “S”; determining the specificity of the reaction, “L”-determining the group responsible for creating the covalent or tight link between compound and target molecule and “A”; determining the group that can be specifically altered.

While “S”, “L” and “A” could be different entities within the compound, they could share identical functions, either as couples or all three together.

In the following examples, different “SLA” components will be illustrated.

The specificity-determining part (the “S”-part) of the compound consists of a functional group or an assemblage of functional groups comprising a chemical moiety interacting with a particular conformation of the target (e.g. the active site of an enzyme). Due to this interaction, the complete compound is brought in close contact with the target allowing the linking being established at reasonable concentrations of the compound. It is well known that increasing concentrations of the compound will decrease the specificity. Thus the “S”-part of the compound should interact with its target under physiologically relevant concentrations. In some situations the compound “S”-part will be able to discriminate the active from the inactive target. Meaning that certain compounds (e.g. drugs) will only target active forms of proteins or, more rarely, others will only target inactive proteins. In other situations, the conformation of the target protein(s), whereby with a reactive functionality or one that requires activation, the predominant reaction will be at the active site. The compound also contains a chemically reactive group (“L”-part) which reacts with a functionality present in the target protein. The link between said compound and its target is most ideally of covalent nature. However, any binding which is sufficiently strong and resistant against all chemical and/or enzymatic treatments, against solvents and buffers used in all chromatographic steps and against all other steps used in the entire sorting procedure could be considered. Such non-covalent, but sufficiently strong binding can for example be formed between coplanar cys-hydroxyl groups and boronic acid derivatives. The “L”-part could be embedded in the “S”-part of the compound as for instance for the enzyme suicide inhibitors such as penicillin, 5-fluorouracil, or the caspase-1 inhibitor. The specificity-determining group and the linking group should not necessarily be present in the same moiety, but could be spatially separated in the compound structure. This is illustrated in example 1.4 where the “S”-part and “L”-part contact different surfaces at the target protein. Such chemically reactive group can be a photo-activatable group such as a diazoketone, arylazide, arylketone, arylmethylhalide, etc. any of which can bind non-selectively to a target protein, but which is transferred by the “S”-part at a specific site of the target protein. Such chemically reactive group can consist of a functional group with higher selectivity. Selectivity for amino-groups such as amidates, succinic acid anhydride and the like; for SH-groups such as methylmaleimide or acetylhalides and the like. Such chemically reactive groups may form links which can be broken afterwards. For instance, bonds formed between maleic acid anhydride and amino-groups may be broken by acid treatment. Such links between the “L”-part and the target protein may be formed by enzymatic catalysis. For instance, links between a glutamine side chain on the target and a lysine εNH₂-group on the compound could be formed by the action of a transglutaminase.

In particular embodiments, the biological target molecule is a polypeptide, a nucleic acid, a carbohydrate, a nucleoprotein, a glycopeptide or a glycolipid, preferably a polypeptide, which may be, for example, an enzyme, a hormone, a transcription factor, a receptor, a peptide ligand for a receptor, a growth factor, an immunoglobulin, a steroid receptor, a nuclear protein, a signal transduction component, an allosteric enzyme regulator, and the like. The biological target can also be a class or family of polypeptides, nucleic acids, carbohydrates, glycopeptides, or glycolipids, preferably a class of proteins such as hydrolases, dehydrogenases, ligases, transferases and proteins that bind to each other or to other biological structures.

The term “altering” or “altered” or “alteration” as used herein in relation to a compound-target complex (e.g. a drug-protein interaction), refers to the introduction of a specific modification in the compound (e.g. a drug), with the clear intention to change the chromatographic behaviour of such a compound-target complex containing said altered compound. Usually the alteration is in the “A” part of the compound (alteration part) but the alteration can also take place in the “S or L” part of the compound (specificity or linking part). Such alteration can be a stable chemical or enzymatical modification. Such alteration can also introduce a transient interaction with a molecule. Typically an alteration will be a covalent reaction, however, an alteration may also consist of a complex formation between the compound bound on the target, provided this complex is sufficiently stable during the chromatographic steps. Typically, an alteration results in a change in hydrophobicity or net charge such that the altered compound-target migrates differently from its unaltered version in revered phase chromatography. Alternatively, an alteration results in a change in the net charge of a compound-target complex, such that the altered compound-target complex migrates different from its unaltered version in an ion exchange chromatography, such as an anion exchange or a cation exchange chromatography. Alternatively, a specific change in the net charge of a compound-target complex may be equally exploited by electrophoretic systems, more particularly by capillary electrophoresis. Also the alteration may be the cleavage of a part of the drug-target complex, for example the “A” part of the drug-target complex. Also, an alteration may result in any other biochemical, chemical or biophysical change in a compound-target complex such that the altered compound-target complex migrates different from its unaltered version in a chromatographic separation. The term “migrates differently” means that a particular altered compound-target complex elutes at a different elution time in run 2 with respect to the elution time of the same non-altered compound-target complex in run 1. Such alterations could induce either a forward or backwards shift of the sorted complex in the secondary run. The alteration step should be more specific for the compound-target complex and should not take place on more than one or on more than a limited set of peptides which do not carry the compound. In this case the altered compound-target complex could be distinguished from the altered peptides by differential analysis. Preferably, the alteration step is highly specific for the compound-target complex and does not take place on any other peptide that does not carry the compound.

Altering can be obtained via a chemical reaction or an enzymatic reaction or a combination of a chemical and an enzymatic reaction of the compound. A non-limiting list of chemical reactions includes alkylation, acetylation, nitrosylation, oxidation, hydroxylation, methylation, reduction, hydrolysis (basic or acid) and the like. A non-limiting list of enzymatic reactions includes treating the compound-target complex with phosphatases, acetylases, glycosidases, specific proteinases or other enzymes which modify co- or post-translational modifications present on compounds. The chemical alteration can comprise one chemical reaction, but can also comprise more than one reaction such as for instance two consecutive reactions in order to increase the alteration efficiency. Similarly, the enzymatic alteration can comprise one or more enzymatic reactions. Such alteration is applied in between two chromatographic separations of the same type.

The resulting altered product is ideally a peptide carrying an altered molecule (a tag) at the site of the original covalent or tight bond. Ideally such a tag should be small and contain a limited number of atoms in order to allow an easy and accurate analysis and identification. More ideally, although not absolutely necessary, such tag should contain a functional group which can be labeled either with heavy or light stable isotopes facilitating quantitative differential analysis by mass spectrometry.

The term ‘stably interacts’ refers to the interaction between a compound (e.g. a drug or drug derivative) added to a complex mixture of molecules (e.g. a complex protein mixture or a protein peptide mixture). Said interaction is strong enough for the isolation of a partner for said compound, in other words a target molecule for said compound. The interaction is sufficiently stable during the two chromatographic separations. In a particular embodiment said interaction is a covalent interaction.

The same type of chromatography means that the type of chromatography is the same in both the initial separation and the second separation. The type of chromatography is for instance in both separations based on the hydrophobicity of the molecules (e.g. peptides) and compound-molecule complexes. Similarly, the type of chromatography can be based in both steps on the charge of the molecules (e.g. peptides) and the use of ion-exchange chromatography or capillary electrophoresis. In still another alternative, the chromatographic separation is in both steps based on a size exclusion chromatography or any other type of chromatography.

The first chromatographic separation, before the alteration, is hereinafter referred to as the “primary run” or the primary chromatographic step” or the “primary chromatographic separation” or “run 1”. The second chromatographic separation of the altered fractions is hereinafter referred to as the “secondary run” or the “secondary chromatographic step” or the “secondary chromatographic separation” or “run 2”.

In a preferred embodiment of the invention the chromatographic conditions of the primary run and the secondary run are identical or, for a person skilled in the art, substantially similar. Substantially similar means for instance that small changes in flow and/or gradient and/or temperature and/or pressure and/or chromatographic beads and/or solvent composition is tolerated between run 1 and run 2 as long as the chromatographic conditions lead to the same or predictable elution of the unaltered molecules in run 2 and to an elution of the altered compound-target complexes (e.g. altered drug-protein or altered drug-peptide complexes) that is predictably distinct from the unaltered molecule-target complexes and this for every fraction collected from run 1. Altered compound-target complexes have a different chromatographic behaviour in run 2. The alteration induces a shift of the altered compound-target complexes. Due to this shift the altered compound-target complexes elute at a different positioning run 2, as compared to run 1, and consequently said complexes can be isolated and identified (see further herein).

In a particular example were protein targets of a particular compound are sought to be determined, after the addition of said compound to a protein peptide mixture, and separating said treated protein peptide mixture into fractions via a primary chromatographic step, the current invention requires that the alteration of compound-peptide complexes is effective in each of the peptide fractions from the primary run. In a fraction derived from said primary run (in a first chromatographic step) peptide and unaltered compound-peptide complexes can be found. Thus, in each fraction obtained from the primary chromatographic step, the altered compound-peptide complexes have to migrate distinctly from the unaltered compound-peptide complexes in the secondary chromatographic step. The alteration of the compound part of the compound-peptide complexes induces a shift in the elution of said altered compound-peptide complex. Depending on the type of applied alteration, the shift may be caused by a change in the hydrophobicity, the net charge and/or the affinity for a ligand (e.g. a metal ion) of the altered compound-peptide complexes. This shift is called δp and is specific for every individual altered compound-peptide complex. In the example of a change in hydrophobicity, δp-values can be expressed as changes in the hydrophobic moment, or as a percentage of organic solvents in chromatographic runs, but most practically in time units under given chromatographic/electrophoretic conditions. Thus δp is not necessary identical for every altered compound-peptide complex and lies in-between δₘₐₓ and δₘᵢₙ. δp is affected by a number of factors such as the nature of the induced alteration, the nature of the column stationary phase, the mobile phase (buffers, solvents), temperature and others. All δp values taken together delineate the extremes of δₘₐₓ and δₘᵢₙ. Given t₁ and t₂, the times delineating the beginning and the end of the interval of the shifted altered compound-peptide complexes, and t₃ and t₄, the times enclosing the fraction taken from the primary run, then δₘᵢₙ (the minimal shift) will be determined by t₃−t₂, while δₘₐₓ (the maximal shift) will be determined by t₄−t₁. Window W₁ is the fraction window in which the unmodified peptides elute in the secondary run w₁=t₄−t₃. Window w₂ is the window in which the altered compound-peptide complexes will elute w₂=t₂−t₁. Thus: δₘᵢₙ=t₃−t₂; δₘᵢₙ=t₄−t₁; w₁=δₘₐₓ+t₁−δₘᵢₙ−t₂ and w₂=t₂−t₁=δₘₐₓ−δₘᵢₙ−w₁. Important elements in the sorting process are: δₘᵢₙ, delineating the distance between the unaltered and the least shifted of the altered compound-peptide complexes in a given fraction and w₂, the time-window in which altered compound-peptide complexes are eluted. The word ‘sorted’ is in this invention equivalent to the word ‘isolated’. δₘᵢₙ has to be sufficient to avoid that altered compound-peptide complexes elute within window w₁ (and as such would overlap with the unaltered compound-peptide complexes), and this rule should apply for every fraction collected from the primary run. Preferentially δₘᵢₙ should be w₁ or larger in order to minimize overlap between altered and unaltered compound-peptide complexes. For instance, if w₁=1 minute, min should by preference be 1 minute or more. Avoiding overlap or co-elution of altered compound-peptide complexes improves the possibility of identifying an optimal number of individual altered compound-peptide complexes. From this perspective, the size of window w₂ has an impact on the number of altered compound-peptide complexes that can be identified. Larger values of w₂ result in a decompression of the altered compound-peptide complex elution time, providing a better isolation of altered compound-peptide complexes and a better opportunity for analysis by gradually presenting the targets (altered compound-peptide complexes) for identification to analysers such as mass spectrometers. While window w₂ may be smaller than w₁, in a preferred embodiment, w₂ will be larger than w₁. For instance if w₁=1 minute, w₂ can be 1 minute or more. It is preferred that the size of w₂, and the value of δₘᵢₙ and δₘₐₓ are identical or very similar for every fraction collected from the primary run. It is however self-evident that minor contaminations of unaltered compound-peptide complexes in the elution window of the altered compound-peptide complexes is not preferred, but it is acceptable. Manipulation of the values of δₘᵢₙ, δₘₐₓ and w₂ to obtain optimal separation of the altered compound-peptide complexes from the unaltered compound-peptide complexes in each primary run fraction is part of the current invention and comprises, among others, the right combination of the compound selected for alteration, the type of alteration, and the chromatographic conditions (type of column, buffers, solvent, etc.). While the aspects of the hydrophilic shift have been worked out herein above, a similar description could also be provided where a hydrophobic shift was induced in order to separate the altered compound-peptide complexes from the non-altered compound-peptide complexes. Here t₃ and t₄ define window w₁ in which the unaltered compound-peptide complexes elute, while t₅ and t₆ define the window w₂ in which the altered compound-peptide complexes elute. The maximum hydrophobic shift δₘₐₓ=t₆−t₃, the minimum shift=t₅−t₄. It will be appreciated that similar calculations for conditions in which fractions are pooled may be used.

It is obvious for a person skilled in the art that the same approach can be applied to isolate compound-peptide complexes with for instance ion exchange chromatography or other types of chromatography.

Thus in case of a complex mixture of peptides (e.g. a protein peptide mixture) in which the compound is only linked to one target peptide or to a limited number of target peptides, while the vast majority of peptides is not conjugated to the compound, then the sorting process is as follows. The total peptide mixture is first separated in the primary chromatographic step. The eluting peptides are collected in an appropriate number of fractions. Then, the alteration step is carried out, for example on the ‘A’-part of the compound-peptide complexes present in each collected fraction. In principle every fraction is subjected to a second chromatographic step. Peptides linked to the compound (so called compound-peptide mixtures) will be altered and show a chromatographic shift. Peptides not linked to the compound will elute in the same predictable position during run 2 with respect to run 1. Since every fraction of run 1 occupies only a fragment of the total separation protocol of run 2, we can combine multiple fractions of run 1, for sorting in run 2. The fractions are combined in such a way, that the sorted peptides (here the compound-peptide complexes) do not overlap with the non-altered peptides of neighbouring fractions. Thus in yet another embodiment, the invention is directed to the use of a sorting device that is able to carry out the method of the invention. As a non-limited example were the molecules of the invention are proteins or peptides, the method may comprise two consecutive chromatographic steps: a primary chromatographic step using for example a protein peptide mixture (to which a compound, comprising a functional group that can be altered, with a specificity for a particular peptide or class of peptides has been added) which divides said mixture into fractions, and a second chromatographic step that is performed after the specific chemical and/or enzymatic alteration of at least one compound-peptide complex present in the fractions. As described herein, the term “peptide sorter” refers to a device that efficiently separates the altered compound-peptide complexes from the non-altered complexes. In a preferred aspect, identical or very similar chromatographic conditions are used in the two chromatographic steps such that during the second run the non-altered compound-peptide complexes stay at their original elution times and the altered compound-peptide complexes are induced to undergo a shift in the elution time. Additionally in another preferred aspect we assume that the alteration of compound-peptide mixtures occurs close to completeness. As described herein, the use of for example a peptide sorter particularly refers to the pooling of fractions obtained after run 1 and the optimal organisation of the second chromatographic step (e.g., the step in which the altered compound-peptides complexes are separated from the non-altered complexes to speed up the isolation of the altered compound-peptide complexes out of each of the run 1 fractions). One approach to isolate and identify altered compound-peptide complexes isolated from a protein peptide mixture, is to independently collect every fraction from the primary chromatographic separation, to carry out the chemical and/or enzymatic alteration on the compound-peptide complexes in each of the fractions and to rerun every fraction independently in the same chromatographic conditions and on the same or substantially similar column. Subsequently the altered compound-peptide complexes of each independently run secondary run are collected and passed to an analytical instrument such as a mass spectrometer. However, such approach requires a considerable amount of chromatography time and occupies important machine time on the mass spectrometer. In order to obtain a more efficient and economic use of both the chromatographic equipment and the mass spectrometer, the present invention provides the use of peptide sorters allowing the pooling of several fractions of the primary chromatographic separation while avoiding elution overlap between altered compound-peptide complexes originating from different fractions, and between altered compound-peptide complexes from one fraction and peptides from one or more other fractions. In each fraction obtained from the primary chromatographic step, altered compound-peptide complexes elute distinct from the unaltered complexes. When several fractions of the primary run are combined (pooled), then it is important that during the second run with the pooled fractions, the sorted altered compound-peptide complexes from one selected fraction do not co-elute with the (unaltered) peptides of one of the previous fractions. The choice of the number of pools will among others depend on (I) the interval shift δp induced by the chemical and/or enzymatic alteration, ii) the elution window of the fractions collected from the primary chromatographic separation and iii) the need to optimise the chromatography time and the analysis time. The current invention also provides the use of a parallel column sorter. With a parallel column sorter, the method based on a single column is executed with a number of columns operating in parallel (i.e., synchronously). The parallel sorter contains a number of identical columns which are run in exactly the same conditions (flow rate, gradient, etc.). A parallel column sorter is most conveniently a device where 2, 3, 4 or more columns perform a secondary chromatographic run at the same time in substantially similar conditions (flow rate, gradient, etc.) and wherein the exit of the parallel sorter is directly connected with an analyzer. A parallel column sorter divides the chromatographic separation time which is normally needed for a series of serial single columns by approximately the number of columns which are used in said parallel sorter. The advantage of using a parallel column sorter is not only that the overall compound-peptide complexes sorting time can be significantly reduced, but also that there are a limited number of dead intervals between the selection of altered compound-peptide complexes from the altered fractions so that the detection of the altered compound-peptide complexes can occur in a continuous manner. In another aspect of the invention, a multi-column peptide sorter can be used. Such a multi-column peptide sorter is created and essentially exists of a number of parallel column sorters that are operating in a combined parallel and serial mode. Such parallel sorter essentially comprises y times a set of z columns, wherein the z columns are connected in parallel. In a non-limiting example, a multi-column sorter where y=3 and z=3 is a nine-column sorter. Such a nine-column sorter operates with three sets of each time three columns connected in parallel. The three parallel column sets are designated as A, B, and C. The individual columns of A are designated as I, II, and III; the individual columns of B are designated as I′, II′; and III′; and the individual columns of C are designated as I″, II″ and III″. One set of parallel columns operates with a delay (named θ) versus the previous set. Therefore, the parallel sorter B starts with a delay of θ with respect to the parallel sorter A, and the parallel sorter C starts with a delay of θ after the start of the parallel sorter B, and with a delay of 2θ after the start of the parallel sorter A. It is important to note that in the multi-column sorter, only one run 1 fraction of altered compound-peptide complexes is processed at a given time per column. Thus, in the example of a nine-column sorter, nine fractions of altered compound-peptide complexes are processed simultaneously. This differs from the two previous described sorters (i.e., a one column peptide sorter and a parallel sorter) where several altered fractions are strategically pooled and loaded simultaneously. As only one fraction of altered compound-peptide complexes is processed at the time on the multi-column sorter, the control of the flow rate accuracy (i.e., in the secondary chromatographic step) is not as important as in the previous sorters. Another advantage of the multi-column sorter is that it is well adapted to separate altered compound-peptide complexes from non-altered complexes in cases where the chromatographic shift of altered compound-peptide complexes varies significantly throughout the different fractions. It will be clear to those skilled in the art that other combinations of parallel and serial columns can lead to similar results. The choice of the number of columns, their arrangement and the fractions loaded on the columns will among others depend on i) the interval δp induced by the chemical or enzymatic alteration, ii) the elution window of the fractions collected from the primary chromatographic separation and iii) the need to optimise the chromatography time and the analysis time. It will further be clear to a person skilled in the art that peptide sorters that carry out the method of the current invention could also be performed in a fully automated manner, using commercially available auto-injectors, HPLC-equipment and automated fraction collectors. Therefore, the present examples of peptide sorters should not be considered as exhaustive. Several variants, including electrophoretic and ion-exchange chromatography systems, are equally feasible. The illustrative embodiment further provides a system for performing the above-described method of proteome analysis in a selective and efficient manner. As discussed, a primary chromatographic column performs an initial separation of the complex peptide mixture. The primary chromatographic column separates the complex peptide mixture into at least two fractions under a defined set of conditions. For example, the primary chromatographic column separates the protein peptide mixture by eluting the column with a predetermined solvent gradient and a predetermined flow rate. The fractions resulting from the primary chromatographic separation may be strategically pooled to combine a plurality of fractions having distinct elution times into a plurality of pooled fractions, as described above. The pooled fractions may be subsequently altered to result in a set of altered peptides and a set of non-altered peptides for each fraction. According to an alternate embodiment, the fractions are first altered using the methods described above and then strategically pooled into a set of pooled fractions, wherein each fraction in a pooled fraction comprises a set of altered compound-peptide complexes and a set of non-altered compound-peptide complexes. In a secondary chromatographic separation, the altered complexes are separated from the unaltered complexes. The isolated targets (=the altered compound-peptide complexes) may then be analyzed to identify a protein.

In another embodiment the present invention further provides a method to identify the isolated targets (=the altered compound-target complexes). In a particular embodiment the identification of the targets can be carried out by a mass spectrometric approach. In another particular embodiment where the target molecules are proteins or peptides said identifying step is performed by a method selected from the group consisting of: a tandem mass spectrometric method, Post-Source Decay analysis, measurement of the mass of the peptides, in combination with database searching. In yet another particular embodiment the identification method based on the mass measurement of the peptides is further based on one or more of the following: (a) the determination of the number of free amino groups in the target peptides; (c) the knowledge about the cleavage specificity of the protease used to generate the protein peptide mixture; and (d) the grand average of the hydropathicity of the target peptides.

In a particular embodiment the targets are proteins or peptides and therefore the method of the invention is further coupled to a peptide analysis. The present invention therefore further provides a method to identify target peptides and their corresponding proteins. In a preferred approach the analysis of altered drug-peptide complexes is performed with a mass spectrometer. However, drug-peptide complexes can also be further analysed and identified using other methods such as electrophoresis, activity measurement in assays, analysis with specific antibodies, Edman sequencing, etc. An analysis or identification step can be carried out in different ways. In one way, altered drug-peptide complexes (the tagged peptides) eluting from the chromatographic columns are immediately directed to the analyzer. In an alternative approach, altered drug-peptide complexes are collected in fractions. Such fractions may or may not be manipulated before going into further analysis or identification. An example of such manipulation consists of a concentration step, followed by spotting each concentrate on for instance a MALDI-target for further analysis and identification. In a preferred embodiment altered drug-peptide complexes are analysed with high-throughput mass spectrometric techniques.

The information obtained is primarily the mass of the tagged peptide(s). This mass is the sum of the mass of the peptide and the mass of the tag (the altered compound component). Since the latter mass is known from the alteration reaction, this tag mass can be subtracted from the total mass of the tagged peptide resulting in a peptide mass which will be the basis in further searching algorithms.

Generally, a mass information is not sufficient for unambiguous peptide identification. Therefore the tagged peptides (=the altered compound-peptide complexes) are further fragmented. This is often done by collision-induced dissociation (CID) in an electrospray instrument or MALDI and is generally referred to as MS/MS or tandem mass spectrometry. Manual or automated interpretation of these MS/MS spectra leads to the assignment of sequence tags and to the identification of the peptide sequence tags and to the location of the tag. Protein identification software which can be used in the present invention to compare the experimental fragmentation spectra of the tagged peptide with amino acid sequences stored in peptide databases. Such algorithms are available in the art.

One such algorithm, ProFound, uses a Bayesian algorithm to search protein or DNA database to identify the optimum match between the experimental data and the protein in the database. ProFound may be accessed on the World-Wide Web at <http//prowl.rockefeller.edu> and <http//www.proteometrics.com>. Profound accesses the non-redundant database (NR). Peptide Search can be accessed at the EMBL website. See also, Chaurand P. et al. (1999) J. Am. Soc. Mass. Spectrom 10, 91, Patterson S. D., (2000), Am. Physiol. Soc., 59-65, Yates J R (1998) Electrophoresis, 19, 893). MS/MS spectra may also be analysed by MASCOT (available at http://www.matrixscience.com, Matrix Science Ltd. London).

Any mass spectrometer may be used to analyze the altered drug-peptide complexes. Non-limiting examples of mass spectrometers include the matrix-assisted laser desorption/ionization (“MALDI”) time-of-flight (“TOF”) mass spectrometer MS or MALDI-TOF-MS, available from PerSeptive Biosystems, Framingham, Mass.; the Ettan MALDI-TOF from AP Biotech and the Reflex III from Brucker-Daltonias, Bremen, Germany for use in post-source decay analysis; the Electrospray Ionization (ESI) ion trap mass spectrometer, available from Finnigan MAT, San Jose, Calif.; the ESI quadrupole mass spectrometer, available from Finnigan MAT or the QSTAR Pulsar Hybrid LC/MS/MS system of Applied Biosystems Group, Foster City, Calif. and a Fourrier transform mass spectrometer (FTMS) using an internal calibration procedure (O'Connor and Costello (2000) Anal. Chem. 72, 5881-5885).

Alternatively, tagged peptide ions can decay during their flight after being volatilised and ionised in a MALDI-TOF-MS. This process is called post-source-decay (PSD). Knowing the peptide sequences stored in peptide sequence databases, it is possible to deduce parts of or the total sequence from such PSD spectra. As above, this analysis can be done manually or by using computer algorithms which are well known in the field. One such algorithm is for instance the MASCOT program.

In a particular embodiment additional sequence information can be obtained in MALDI-PSD analysis when the alfa-NH₂-terminus of the target peptides is altered with a sulfonic acid moiety group. Target peptides carrying an NH₂-terminal sulfonic acid group are induced to particular fragmentation patterns when detected in the MALDI-TOF-MS mode. The latter allows a very fast and easy deduction of the amino acid sequence.

Alternatively, tagged peptides could also be analyzed by conventional Edman-degradation and the obtained amino acid sequence compared to sequences stored in protein or genomic sequence databases. In case the compound itself is a peptide, then Edman-sequencing will generate at each cycle a double amino acid identification, until the degradation reaches the residue of one of the chains which is involved in the isopeptide linkage.

Once, a tagged peptide is unambiguously identified by MS-based fragmentation analysis, further similar experiments may then simply use its total mass. This is for instance the case when activity-based protein profiling of a specific target is carried out on a large number of samples. Indeed, the amount of tagged peptide formed will be dependent on the accessibility and specific reactivity of the target. Once the specific tagged peptide fully characterized in terms of total mass and elution times, it suffices to select the tagged peptide based on its exact mass. A peptide mass can be sufficiently accurately measured with a Fourrier transform mass spectrometer (FT-MS) or using recently developed MALDI based time of flight machines. Such machines are for instance constructed by Bruker-Daltonics, Bremen, Germany (Ultraflex).

If the accuracy by which the mass of the tagged peptide can be measured is not sufficiently discriminative, then additional information can be generated. For example, the elution time by which a given peptide elutes during chromatography, is a parameter which is totally independent of the peptide mass.

Thus the probability is low that two or more peptides, with identical masses or with masses falling within the error range of the mass measurements, also elute with identical or very similar retention times during chromatography. Since the retention time of a peptide during RP-chromatography is primarily related to its overall hydrophobicity, the Grand Average hydropathicity (GRAVY) index, which can be calculated using hydropathicity values given to every natural amino acid. Thus the mass together with the GRAVY index are two independent parameters highly characteristic for a given peptide.

In another embodiment the method of determining the identity of the parent protein by only accurately measuring the peptide mass of at least one target peptide can be improved by further enriching the information content of the selected target peptides. As a non-limiting example of how information can be added to the target peptides, the free NH₂-groups of these peptides can be specifically chemically changed in a chemical reaction by the addition of two different isotopically labelled groups. As a result of this change, said peptides acquire a predetermined number of labelled groups. Since the change agent is a mixture of two chemically identical but isotopically different agents, the target peptides are revealed as peptide twins in the mass spectra. The extent of mass shift between these peptide doublets is indicative for the number of free amino groups present in said peptide. To illustrate this further, for example the information content of target peptides can be enriched by specifically changing free NH₂-groups in the peptides using an equimolar mixture of acetic acid N-hydroxysuccinimide ester and trideuteroacetic acid N-hydroxysuccinimide ester. As the result of this conversion reaction, peptides acquire a predetermined number of CH₃—CO (CD₃-CO) groups, which can be easily deduced from the extent of the observed mass shift in the peptide doublets. As such, a shift of 3 amu's corresponds with one NH₂-group, a 3 and 6 amu's shift corresponds with two NH₂-groups and a shift of 3, 6 and 9 amu's reveals the presence of three NH₂-groups in the peptide. This information further supplements the data regarding the peptide mass and/or the knowledge that the peptide was generated with a protease with known specificity.

The use of the mass of a sorted tagged peptide as the sole peptide/protein identification criterion becomes important and reasonable once said tagged-peptide has been fully identified (previously) by other means such as those described above.

For instance, once a tagged peptide has been fully identified by MS-fragmentation analysis and database searching, further identification can be based on the accurately measured mass of the tagged peptide, without repeating each time the MS/MS-analysis.

Thus the expression levels or the activity and expression levels of a biological target or different biological targets present in a multitude of samples means more than one, preferably more than five, more preferably more than one hundred and more preferably more than thousand and more preferably a number typically encountered during high-throughput analysis. A highly complex mixture of proteins refers to cell lysates, cell fractions, tissues, biological fluids and the like as they are described below.

In cases where the invention leads to the identification of the members of a class of biological targets, then the mass of every tagged peptide could be representative of its corresponding biological target protein and the invention would allow a global analysis of levels or levels and/or activities of each member of the family. For instance, the use of FSBA to target ATP-binding proteins and in particular the kinase families, can lead to a number of tagged peptides. Each of these tagged peptides will carry the same tag but will be otherwise distinct by the peptide-moiety. Thus each kinase level and/or activity will be reflected by the specific peptide mass in the tagged peptide. Relative quantification of every tagged peptide will provide a global profile of levels and/or activities of the members of a family of biological targets.

Although absolute quantification of peptides by mass spectrometry is very difficult, MS-based techniques are suitable for comparative analysis.

Thus in another embodiment a method is provided to determine the relative amount of the level and/or activity of at least one target protein in more than one sample comprising proteins, comprising the steps of (a) the addition of a compound comprising a first isotope to a first sample comprising peptides wherein said compound stably interacts with at least one peptide forming a compound-peptide complex; (b) the addition of a compound comprising a second isotope to a second sample comprising peptides wherein said compound stably interacts with at least one peptide forming a compound-peptide complex; (c) combining the protein peptide mixture of the first sample with the protein peptide mixture of the second sample; (d) separating the combined protein peptide mixtures into fractions of peptides via chromatography; (e) chemically, or enzymatically, or chemically and enzymatically, altering said compound present on at least one compound-peptide complex in each fraction; (f) isolating the altered compound-peptide complexes out of each fraction via chromatography, wherein the chromatography is performed with the same type of chromatography as in step (d); (g) performing mass spectrometric analysis of the isolated altered compound-peptide complexes; (h) calculating the relative amounts of the altered compound-peptide complexes in each sample by comparing the peak heights of the identical but differentially, isotopically labelled altered compound-peptide complexes, and (i) determining the identity of said peptides in the altered compound-peptide complexes and their corresponding proteins.

To compare the level and/or activity of the targets in two different samples, differential mass labeling can be used. Therefore, the compound-peptide complexes (the tagged peptides) of the first sample can be labeled with “light” atoms, while the tagged peptide of the second sample will be labeled with “heavy atoms”. Labeling can for instance be carried out by the use of a compound that carries an isotopic label. Before the primary chromatographic run the compound-peptide target complexes of both samples will be mixed. The “light” and “heavy” components will elute or migrate in an identical or nearly identical manner during the primary run. Their alteration will also proceed in the same manner. The “light” and “heavy” tagged peptides will elute or migrate in an identical or nearly identical manner and co-transferred to the mass spectrometer. Only during the analysis with the latter, “light” and “heavy” tagged peptide ions will segregate and their relative intensities can be measured. It is important to stress that the discriminating atoms remain attached to the tagged peptide after the alteration step. Thus both the “light” and “heavy” atoms are part of the tag on the tagged peptide.

As couple of light and heavy atoms, one can use H/D, ¹⁶O/¹⁸O, ¹²C/¹³C, ¹⁴N/¹⁵N or any couple of stable isotopes which can be stably incorporated in organic and inorganic compounds. In an alternative embodiment the proteins can be labeled instead of the compounds. The differential isotopic labeling of peptides in a first and a second sample can be done in many different ways available in the art. A key element is that a particular peptide originating from the same protein in a first and a second sample is identical, except for the presence of a different isotope in one or more amino acids of the peptide. In a typical embodiment the isotope in a first sample will be the natural isotope, referring to the isotope that is predominantly present in nature, and the isotope in a second sample will be a less common isotope, hereinafter referred to as an uncommon isotope. Examples of pairs of natural and uncommon isotopes are H and D, O¹⁶ and O¹⁸, C¹² and C¹³, N¹⁴ and N¹⁵. Peptides labeled with the heaviest isotope of an isotopic pair are herein also referred to as heavy peptides. Peptides labeled with the lightest isotope of an isotope pair are herein also referred to as light peptides. For instance, a peptide labeled with H is called the light peptide, while the same peptide labeled with D is called the heavy peptide. Peptides labeled with a natural isotope and its counterparts labeled with an uncommon isotope are chemically very similar, separate chromatographically in the same manner and also ionize in the same way. However, when the peptides are fed into an analyser, such as a mass spectrometer, they will segregate into the light and the heavy peptide. The heavy peptide has a slightly higher mass due to the higher weight of the incorporated, chosen isotopic label. Because of the minor difference between the masses of the differentially isotopically labeled peptides the results of the mass spectrometric analysis of isolated altered compound-peptide complexes will be a plurality of pairs of closely spaced twin peaks, each twin peak representing a heavy and a light altered complex. Each of the heavy complexes is originating from the sample labelled with the heavy isotope; each of the light complexes is originating from the sample labelled with the light isotope. The ratios (relative abundance) of the peak intensities of the heavy and the light peak in each pair are then measured. These ratios give a measure of the relative amount (differential occurrence) of that target (as an isolated altered compound-complex) in each sample. The peak intensities can be calculated in a conventional manner (e.g. by calculating the peak height or peak surface). As herein described above, the altered compound-peptide complexes can also be identified allowing the identification of proteins in the samples. If a protein target for a particular compound is present in one sample but not in another, the isolated altered compound-peptide complexes (corresponding with this protein) will be detected as one peak which can either contain the heavy or light isotope. However, in some cases it can be difficult to determine which sample generated the single peak observed during mass spectrometric analysis of the combined sample. This problem can be solved by double labeling the first sample, either before or after the proteolytic cleavage, with two different isotopes or with two different numbers of heavy isotopes. Examples of labeling agents are acylating agents.

Incorporation of the natural and/or uncommon isotope in peptides can be obtained in multiple ways. In one approach proteins are labeled in the cells. Cells for a first sample are for instance grown in media supplemented with an amino acid containing the natural isotope and cells for a second sample are grown in media supplemented with an amino acid containing the uncommon isotope. In another embodiment the incorporation of the differential isotopes can also be obtained by an enzymatic approach. For instance labeling can be carried out by treating one sample comprising proteins with trypsin in “normal” water (H₂¹⁶O) and the second sample comprising proteins with trypsin in “heavy” water (H₂¹⁸O). As used herein “heavy water” refers to a water molecule in which the O-atom is the ¹⁸O-isotope. Trypsin shows the well-known property of incorporating two oxygens of water at the COOH-termini of the newly generated sites. Thus in sample one, which has been trypsinized in H₂¹⁶O, peptides have “normal” masses, while in sample two, peptides (except for most of the COOH-terminal peptides) have a mass increase of 4 amu's corresponding with the incorporation of two 180 atoms This difference of 4 amu's is sufficient to distinguish the heavy and light version of the altered compound-peptide complexes in a mass spectrometer and to accurately measure the ratios of the light versus the heavy peptides and thus to determine the ratio of the corresponding target peptides/target proteins in the two samples.

Incorporation of the differential isotopes can further be obtained with multiple labelling procedures based on known chemical reactions that can be carried out at the protein or the peptide level. For example, proteins can be changed by the guadinylation reaction with O-methylisourea, converting NH₂-groups into guanidinium groups, thus generating homoarginine at each previous lysine position. Proteins from a first sample can be reacted with a reagent with the natural isotopes and proteins from a second sample can be reacted with a reagent with an uncommon isotope. Peptides could also be changed by Shiff's-base formation with deuterated acetaldehyde followed by reduction with normal or deuterated sodiumborohydride. This reaction, which is known to proceed in mild conditions, may lead to the incorporation of a predictable number of deuterium atoms. Peptides will be changed either at the α-NH₂-group, or ε-NH₂ groups of lysines or on both. Similar changes may be carried out with deuterated formaldehyde followed by reduction with deuterated NaBD₄, which will generate a methylated form of the amino groups. The reaction with formaldehyde could be carried out either on the total protein, incorporating deuterium only at lysine side chains or on the peptide mixture, where both the α-NH₂ and lysine-derived NH₂-groups will be labelled. Since arginine is not reacting, this also provides a method to distinguish between Arg- and Lys-containing peptides. Primary amino groups are easily acylated with, for example, acetyl N-hydroxysuccinimide (ANHS). Thus, one sample can be acetylated with normal ANHS whereas a second sample can be acylated with either ¹³CH₃CO—NHS or CD₃CO—NHS. Also the ε-NH₂ group of all lysines is in this way derivatized in addition to the amino-terminus of the peptide. Still other labelling methods are for example acetic anhydride which can be used to acetylate hydroxyl groups and trimethylchlorosilane which can be used for less specific labelling of functional groups including hydroxyl groups and amines.

In yet another approach the primary amino acids are labelled with chemical groups allowing to differentiate between the heavy and the light peptides by 5 amu, by 6 amu, by 7 amu, by 8 amu or even by larger mass difference. Alternatively, the differential isotopic labelling is carried out at the carboxy-terminal end of the peptides, allowing the differentiation between the heavy and light variants by more than 5 amu, 6 amu, 7 amu, 8 amu or even larger mass differences. Since the methods of the present invention do not require any prior knowledge of the type of target proteins that may be present in the samples, they can be used to determine the relative amounts of both known and unknown target proteins which are present in the samples examined.

The methods provided in the present invention to determine relative amounts of at least one protein target and/or the activity of a protein in at least two samples can be broadly applied to compare protein levels in for instance cells, tissues, or biological fluids, organs, and/or complete organisms. Such a comparison includes evaluating subcellular fractions, cells, tissues, fluids, organs, and/or complete organisms which are, for example, diseased and non-diseased, stressed and non-stressed, drug-treated and non drug-treated, benign and malignant, adherent and non-adherent, infected and uninfected, transformed and untransformed. The method also allows to compare protein target levels or the activity of one or more proteins in subcellular fractions, cells, tissues, fluids, organisms, complete organisms exposed to different stimuli or in different stages of development or in conditions where one or more genes are silenced or overexpressed or in conditions where one or more genes have been knocked-out.

In another embodiment, the methods described herein can also be employed in diagnostic assays for the detection of the presence, the absence or a variation in level of one or more protein targets and/or the activity of a protein or a specific set of proteins indicative of a disease state (e.g., such as cancer, neurodegenerative disease, inflammation, cardiovascular diseases, viral infections, bacterial infections, fungal infections or any other disease). Specific applications include the identification of target proteins which are present in metastatic and invasive cancers, the differential expression of proteins in transgenic mice, the identification of proteins that are up- or down-regulated in diseased tissues, the identification of intracellular changes in cells with physiological changes such as metabolic shift, the identification of biomarkers in cancers, the identification of signalling pathways.

Samples that can be analyzed by methods of the invention include biological samples, such as cell lysates, microsomal fractions, cell fractions, tissues, organelles, etc., and biological fluids including urine, sputum, saliva, synovial fluid, nipple aspiration fluid, amnion fluid, blood, cerebrospinal fluid, tears, ejaculate, serum, pleural fluid, ascites fluid, stool, or a biopsy sample. If the sample is impure (e.g., plasma, serum, stool, ejaculate, sputum, saliva, cerebrospinal fluid, or blood or a sample embedded in paraffin), it may be treated prior to employing a method of the invention, frequently to remove contaminants of the components of interest. Procedures include, for example, filtration, extraction, centrifugation, affinity sequestering, etc. Where the probes do not readily pass through a cellular membrane, intact or permeabilized, or where a lysate is desirable, the cells are treated with a reagent effective for lysing the cells contained in the fluids, tissues, or animal cell membranes of the sample, and for exposing the proteins contained therein and, as appropriate, partially separating the proteins from other aggregates or compounds such as microsomes, lipids, carbohydrates and nucleic acids in the sample. Methods for purifying or partially purifying proteins from a sample are well known in the art (e.g., Sambrook et al., Molecular Cloning: A Laboratory Manual, Cold Spring Harbor Press, 1989, herein incorporated by reference). The samples may come from different sources and be used for different purposes.

Usually, a proteome will be analyzed. By a proteome is intended at least about 20% of total protein coming from a biological sample source, usually at least about 40%, more usually at least about 75%, and generally 90% or more, up to and including all of the protein obtainable from the source. Thus the proteome may be present in an intact cell, a lysate, a microsomal fraction, an organelle, a partially extracted lysate, biological fluid, and the like. The proteome will be a complex mixture of proteins, generally having at least about 20 different proteins, usually at least about 50 different proteins and in most cases 100 different proteins or more. In effect, the proteome is a complex mixture of proteins from a natural source and will usually involve having the potential of having 10, usually 20, or more proteins that are target proteins for a specific compound used to analyze the proteome profile. The sample will be representative of the target proteins of interest. The sample may be adjusted to the appropriate buffer concentration and pH, if desired. One or more compounds, having the structure SLA, may then be added, each at a concentration in the range of about 0.001 mM to 20 mM. After incubating the reaction, generally for a time for the reaction to go substantially to completion, generally for about 1-60 min, at a temperature in the range of about 20-40° C., the reaction may be quenched.

The method of the present invention is useful in supporting the development of new drugs and identifying (new) drug targets. One embodiment of the subject invention is especially useful for rapidly screening a number of drug candidate compounds. The invention is also useful for systematically analyzing a number of compounds that may vary greatly in their chemical structure or composition, or that may vary in minor aspects of their chemical structure or composition. The invention is also useful for optimizing candidate drugs that show the most medicinal promise, meaning binding to a particular, desired target and not to others. The invention can also be used to measure enzymatic activities or biological activities in general or the sum of expression levels and activities of biological molecules in total extracts of tissues, cells, cell organelle and protein complexes. The ability to predict the toxic effects of potential new drugs is crucial to prioritizing compound pipelines and eliminating costly failures in drug development. Toxicogenomics, which deals primarily with the effects of compounds on gene expression patterns in target cells or tissues, is emerging as a key approach in screening new drug candidates because it may reveal genetic signatures that can be used to predict toxicity in these compounds. The current invention focuses on a proteomic approach for the detection of drug targets and hence the method could be designated as toxicoproteomics. The method of the present invention could also be used for the design and optimization of clinical trials. With the method is possible to develop potentially, smaller clinical trials targeting more specific populations that are likely to respond to the drug and that are not likely to develop adverse drug reactions. This, in turn, the use of the method could potentially reduce the cost and time required for clinical trials.

In what follows, a more informative description of several of the different steps of the invention is presented.

I. Preparation of a Protein Peptide Mixture

Protein peptide mixtures originating from a sample comprising proteins for a compound treated sample comprising proteins are obtained by methods described in the art such as chemical or enzymatic cleavage or digestion. In a preferred aspect, the proteins and compound-protein complexes are digested by a proteolytic enzyme. Trypsin is a particularly preferred enzyme because it cleaves at the sites of lysine and arginine, yielding charged peptides which typically have a length from about 5 to 50 amino acids and a molecular weight of between about 500 to 5,000 dalton. Such peptides are particularly appropriate for analysis by mass spectroscopy. A non-limited list of proteases which may also be used in this invention includes Lysobacter enzymogenes endoproteinase Lys-C, Staphylococcus aureus endoproteinase Glu-C (V8 protease), Pseudomonas fragi endoproteinase Asp-N and clostripain. Proteases with lower specificity such as Bacillus subtilis subtilisin, procain pepsin and Tritirachium album proteinase K may also be used in this invention.

Alternatively, chemical reagents may also be used to cleave the proteins into peptides. For example, cyanogen bromide may be used to cleave proteins into peptides at methionine residues. Chemical fragmentation can also be applied by limited hydrolysis under acidic conditions. Alternatively, BNPS-skatole may be used to cleave at the site of tryptophan. Partial NH₂-terminal degradation either using chemically induced ladders with isothiocyanate or using aminopeptidase treatment can be used as well.

II. Chromatography

As used herein, the term “chromatographic step” or chromatography” refers to methods for separating chemical substances and are vastly available in the art. In a preferred approach it makes use of the relative rates at which chemical substances are adsorbed from a moving stream of gas or liquid on a stationary substance, which is usually a finely divided solid, a sheet of filter material, or a thin film of a liquid on the surface of a solid. Chromatography is a versatile method that can separate mixtures of molecules even in the absence of detailed previous knowledge of the number, nature, or relative amounts of the individual substances present. The method is widely used for the separation of chemical molecules of biological origin (for example, amino acids, fragments of proteins, peptides, proteins, phospholipids, steroids etc.) and of complex mixtures of petroleum and volatile aromatic mixtures, such as perfumes and flavours. The most widely used columnar liquid technique is high-performance liquid chromatography, in which a pump forces the liquid mobile phase through a high-efficiency, tightly packed column at high pressure. Recent overviews of chromatographic techniques are described by Meyer M., 1998, ISBN: 047198373X and Cappiello A. et al. (2001) Mass Spectrom. Rev. 20(2): 88-104, incorporated herein by reference. Other recently developed methods described in the art and novel chromatographic methods coming available in the art can also be used. Some examples of chromatography are reversed phase chromatography (RP), ion exchange chromatography, hydrophobic interaction chromatography, size exclusion chromatography, gel filtration chromatography or affinity chromatography such as immunoaffinity and immobilized metal affinity chromatography.

Chromatography is one of several separation techniques. Electrophoresis and all variants such as capillary electrophoresis, free flow electrophoresis etc. is another member of this group. In the latter case, the driving force is an electric field, which exerts different forces on solutes of different ionic charge. The resistive force is the viscosity of the non-flowing solvent. The combination of these forces yields ion mobilities peculiar to each solute. Some examples are sodium dodecyl sulfate polyacrylamide gel electrophoresis (SDS-PAGE) and native gel electrophoresis. Capillary electrophoresis methods include capillary gel electrophoresis, capillary zone electrophoresis, capillary electrochromatography, capillary isoelectric focussing and affinity electrophoresis. These techniques are described in McKay P., An Introduction to Chemistry, Science Seminar, Department of Recovery Sciences, Genentech, Inc. incorporated herein by reference.

III. Buffers

The methods of the invention require compatibility between the separation conditions in the primary run, the reaction conditions in the alteration step, the separation condition in the secondary run and the conditions to analyse the eluting altered compound-peptide complexes in analysers such as mass spectrometers. As mentioned before, the combination of the chromatographic conditions in the primary and secondary run and the chromatographic shifts induced by the alteration reaction is determining the possibility to isolate the altered compound-peptide complexes out of each fraction obtained from a protein peptide mixture in the primary run. As also mentioned before, in a preferred embodiment the chromatographic conditions of the primary run and the secondary run are the same or substantially similar.

In a further preferred embodiment, buffers and or solvents used in both chromatographic steps are compatible with the conditions required to allow an efficient proceeding of the chemical and/or enzymatic reactions in the alteration step in between the two chromatographic steps. In a particular preferred embodiment the nature of the solvents and buffer in the primary run, the secondary run and the alteration step are identical or substantially similar. In a further preferred embodiment said buffers and solvents are compatible with the conditions required to perform a mass spectrometric analysis. Defining such buffers and solvents needs tuning and fine-tuning [and such conditions are not available in the prior art].

For some embodiments of the invention with particular types of altered compound-peptide complexes it is very difficult if not impossible to design one set of identical or substantially similar buffers and/or solvents which can be used throughout the procedure of primary run, alteration step, secondary run and analysis.

For instance, the chemical and/or enzymatic reaction to alter the compound-peptide complexes in the alteration step may request specific reaction conditions which are not compatible with the buffers used in the primary and/or secondary run. In these cases the buffer/solvent conditions in the fractions are changed before the alteration step and/or after the alteration step which changing is performed with methods described in the art such as for example an extraction, a lyophilisation and redisolving step, a precipitation and redisolving step, a dialysis against an appropriate buffer/solvent or even a fast reverse phase separation with a steep gradient.

Another complication may be the composition of the buffer/solvent present in a complex protein mixture or a protein peptide mixture before starting the primary run. Application of a pre-treatment step may request specific buffer/solvent conditions which are not compatible with the buffer/solvent to perform the primary run. Alternatively, the conditions for the preparation/isolation of proteins from their biological source may result in the contamination of the protein mixtures or protein peptide mixtures with compounds which negatively interfere with the compound reaction and/or with the primary run. In these situations the buffer/solvent composition of the protein mixture or the protein peptide mixture is changed to make them compatible with the primary run. Such changing is performed with methods described in the art such as for example an extraction, a lyophilisation and redisolving step, a precipitation and redisolving step, a dialysis against an appropriate buffer/solvent or even a fast reverse phase separation with a steep gradient.

In yet another embodiment of the invention the buffer/solvent of the secondary run is not compatible with performing the analysis of the eluting altered compound-peptide complexes. In such cases, the buffer/solvent in the fractions collected from the secondary run is changed to make the conditions compatible with the analysis with for instance a mass spectrometer. Such changing is performed with methods described in the art such as for example an extraction, a lyophilisation and redisolving step, a precipitation and redisolving step, a dialysis against an appropriate buffer/solvent or even a fast reverse phase separation with a steep gradient. Alternatively, the fractions with the altered compound-peptide complexes can be collected and recombined for a third series of separations, hereinafter referred to as a ternary run. Said ternary run is designed in such a way that the eluting flagged or identification peptides can be analysed with a mass spectrometer.

Equivalents

Those skilled in the art will recognize, or be able to ascertain using no more than routine experimentation, many equivalents to the specific embodiments of the invention described herein. For example chromatography can be substituted in many cases by electrophoresis. Electrophoretic techniques include (capillary) gel electrophoresis, (capillary) electrochromatography, (capillary) isoelectric focussing and affinity electrophoresis.

### EXAMPLES

1. The Identification of Drug Targets 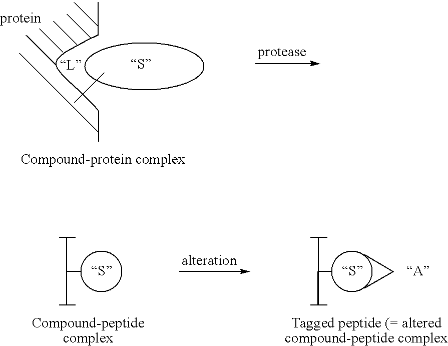
1.1 In a particular compound alll three properties “SLA” reside in the same moiety.
For instance the compound benzoyl-penicilline forms an acyl-enzyme adduct with its target the bacterial DD-aminotranspeptidase. After proteolytic cleavage a penicilloyl-peptide is generated. The alteration step may consist in a conversion of the thioether into a sulfoxide derivative which is more hydrophilic and separates distinctly during chromatographic run 2. 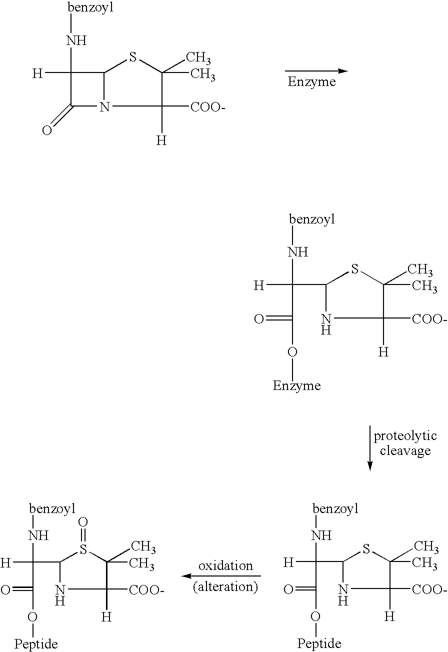
1.2. In a particular compound the “SLA”-moieties are partially separated. The “S”-part interacts with the target molecule. Chemical cross-linking is established by the same group. Thus “S” and “L” are the same. The third “A”-group is altered. 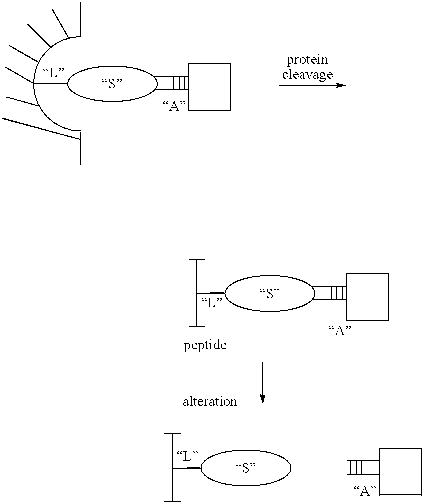
For instance, the molecule could be composed of a Lys-containing peptide which can be cross-linked to Gln-41 of G-actin, through the catalytic action of a transglutaminase such as factor XIIIa (specific labelling of G-actin at Gln-41 with cadaverine or cadaverine-derivatives by zero-length cross-linking with a transglutaminase has been reported previously (Takashi (1988) Biochemistry 27(3): 938). The isopeptide-linkage created between Gln-41 of actin and the Lys-containing peptide is similar.

The sequence of the compound peptide in the one-letter notation is Ac-F-I-E-G-R-A-D-S-K-S-S-COOH has an acetylated free α-NH₂-terminus and a free COOH-terminus. According to our “SLA”-definitions, we distinguish the following functions:

The specificity-determining group (“S”) is composed of the Lys-residue, flanked on both sides by Ser-residues. The Ser-residues and the Asp, incorporated in the COOH-terminal part, further contribute to the hydrophilic character (and therefore solubility) of the final cross-linked peptide. They also contrast with the hydrophobic Phe-Ile cluster, located in the extreme NH₂-terminus, forming a hydrophobic-hydrophilic balance, which will be broken during the alteration step.

The Lys-residue, determining the transglutaminase reaction specificity, is also the residue involved in the zero-length isopeptide formation. Thus here “S” and “L” are the same moieties.

The factor Xa-restriction cleavage site, forms the “A”-part of the compound and is spatially separated from the “S-L”-part. When released by cleavage, the hydrophobic Ac-F-I-E-G-R cargo will separate, leaving a more hydrophilic compound still attached to its target peptide. In the secondary run (run 2), this more hydrophilic peptide will shift in front of the bulk of unmodified peptides.

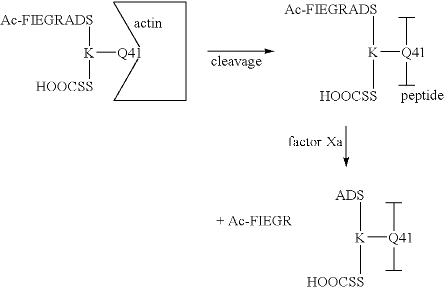

This experiment will be described in detail in examples 1.5 and 1.6
1.3 In a particular compound the three properties “SLA” are more separated: the specificity determining group “S” interacts with the target molecule, chemical cross-linking (“L”) is established by a second group while a third moiety (“A”) is subject to alteration. The resulting tagged peptide still carries the “S” or part of the “S” moiety.

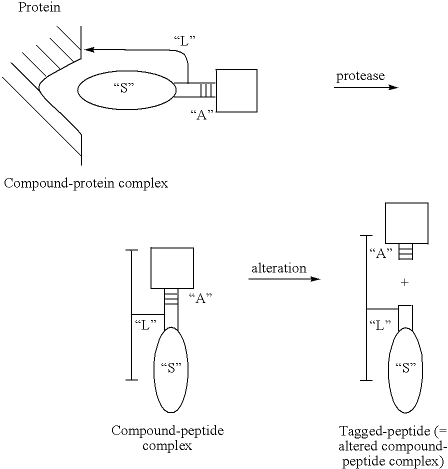

For instance, the molecule could be composed of the caspase-1 inhibitory peptide aldehyde Ac-YVAD-CHO, elongated versus the N-terminal side by, a short peptide carrying the factor Xₐ restriction cleavage site, for instance:

Ac-A-A-I-E-G-R-Y-V-A-D-CHO. While the Y-V-A-D-sequence will direct the molecule to the active site of the caspase-1 type proteases (“S”-group) the COOH-terminus converted into an aldehyde will create the cross-link (“L”-group). The NH₂-terminal part of the molecule can be cleaved off by using factor Xₐ.

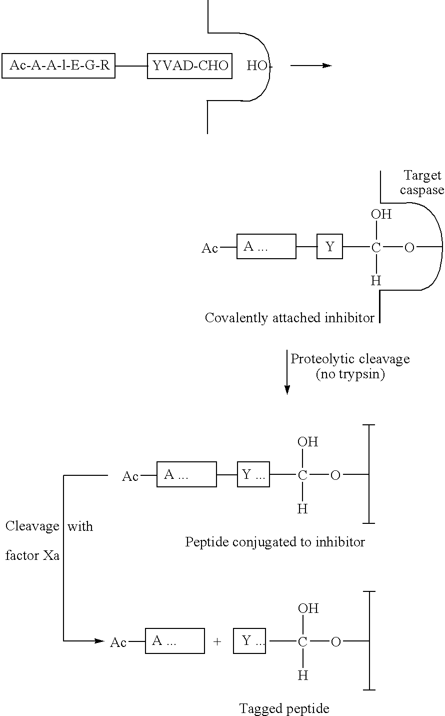

1.4 In one compound the three properties “SLA” are more separated.

The specificity-determining group “S” interacts with the target molecule. Cross-linking is established by another moiety in the molecule reacting with another part of the target. The alteration consists in the separation of the “S”- and the “L”-moieties. The tag now does not contain the “S”-group anymore but the “L”-group or part of the “L”-group.

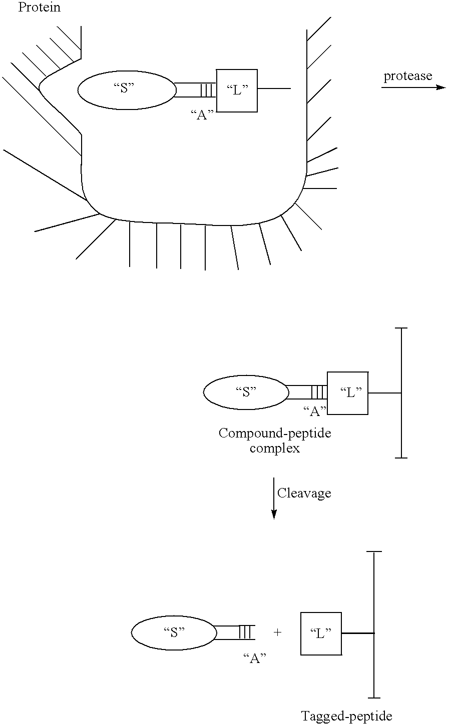

For instance fluorosulfenylbenzoyl adenosine FSBA can form a complex with ATP-binding proteins. FSBA reacts covalently with a lysine derivative located in the active center opposite to the interaction site of the adenosyl-moiety. The FSBA-peptide complex is generated upon proteolytic cleavage. The alteration step consists of an alkaline-induced hydrolysis of the molecule leaving only the sulfenylbenzoyl moiety attached as tag on the peptide. Since FSBA is known to mimic ATP-binding, this method could be used to localize the ATP-binding site(s) in the primary structure of target proteins, to identify ATP-binding proteins and to profile kinase activities in a global cellular context. 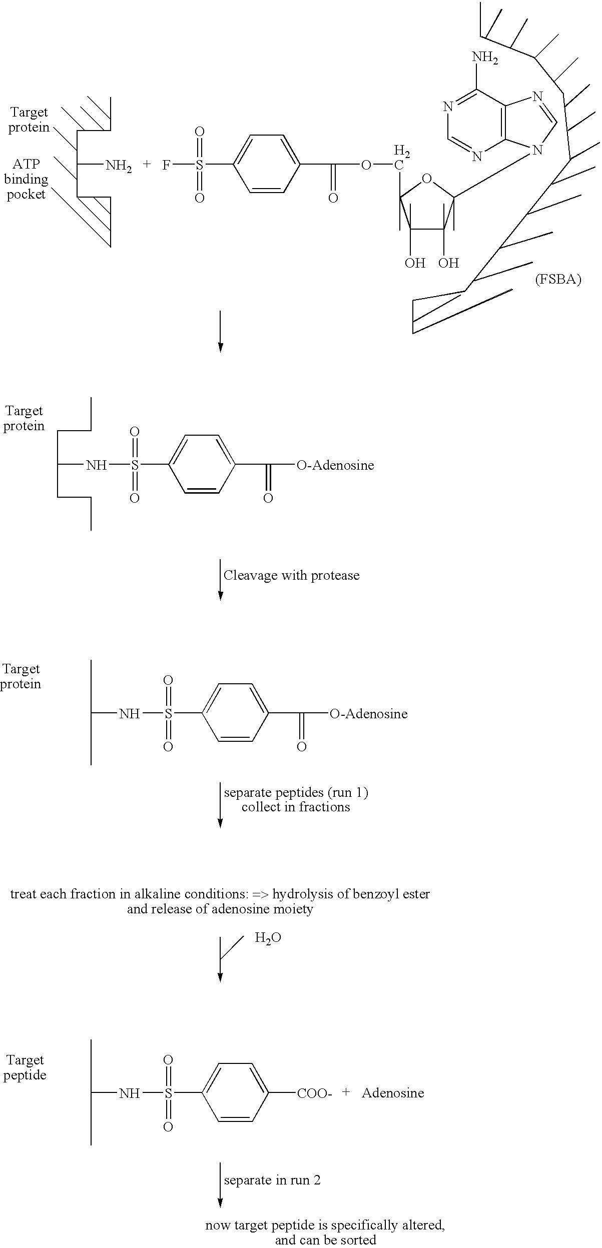
1.5 Identification of the Target Site of a Compound in a Purified Protein

In this example, purified skeletal muscle actin was covalently linked with a synthetic Lys-containing peptide at the actin Gln-41 position. The design and sequence of the synthetic peptide, here referred as “compound peptide or CP” is described in example 1.2.

The CP was incubated overnight at 5 molar excess over 10 nmoles of G-actin in 400 μl of 5 mM Tris-HCl, pH 8.0, 1 mMATP 1 mM Ca Cl₂ and 10 mM β-mercaptoethanol. The isopeptide linkage between the Lys-9 of CP and Gln-41 of actin was formed by catalysis of 0.25 Units of guinea pig liver transglutaminase.

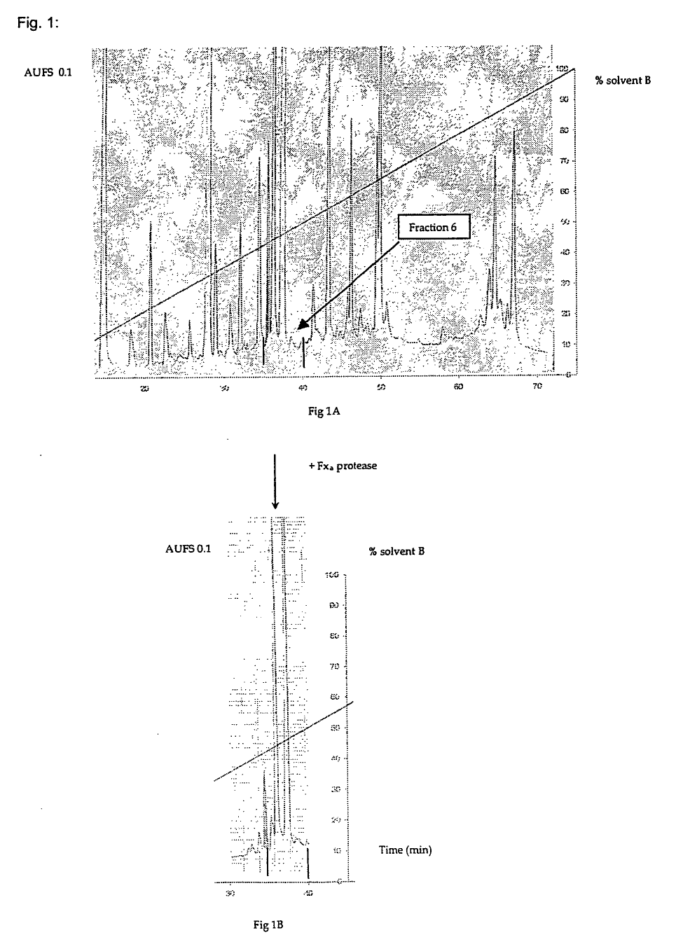

After overnight incubation at 4° C., the mixture was denaturated by boiling for 5 min and further digested with endoproteinase Lys C in the following buffer: 25 mM Tris-HCl pH 8.5 1 mM EDTA with an enzyme/substrate ratio 1/50 by weight. The digestion was carried out during 5 h at 37° C. and stopped by adding trifluoro acetic acid (TFA) to a final concentration of 0.2%. The peptide mixture was centrifuged and loaded (100 μl, corresponding to 84 μg (2 nmol) of actin) on a C-18 reversed-phase column (4,6 mm×250 mm). Peptides were eluted with a linear gradient of acetonitrile (1.4% increase per minute) in 0.1% TFA (for details see FIG. 1A) and recorded by UV-absorption at 214 nm. The peptide elution profile of the endo Lys C digest on the actin-peptide conjugate is shown in FIG. 1A. Peptides were collected in 5 min. or 5 ml fractions and dried by vacuum centrifugation (Savant Instrument). Each fraction was redissolved in 400 μl 40 mM Tris-HCl pH7.3, 50 mM NaCl and treated with 0.12 Units of factor Xa (Promega). After digestion for 3 h at room temperature, TFA was added (final concentration 0.5%) and loaded on the same RP-chromatographic system. Of all the fractions analysed, only fraction 6 showed a shifting (shadow peak) peptide (FIG. 1B).

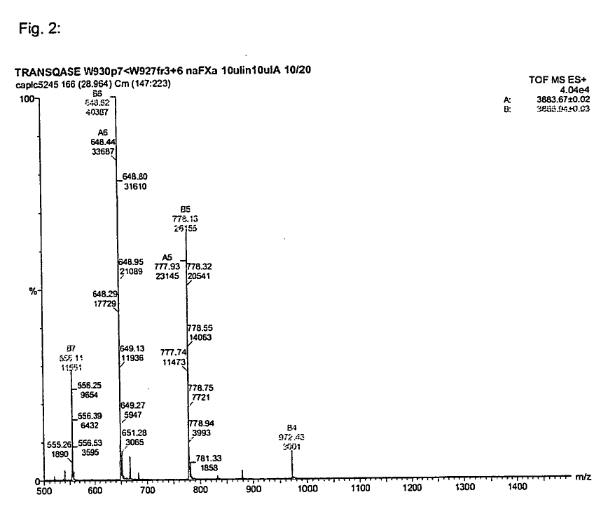

Electrospray-ionization mass spectrometry carried out with a Q-TOF Micromass instrument, confirmed the mass of the cross linked peptide (FIG. 2): Mm abs.: 3883.7 (Mm calc.: 3883.9), corresponding with the 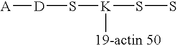
dipeptide.

Edman-degradation further confirmed the sequence of the two cross-linked chains: Cycle 1: Ala; Cycle 2: Gly+Asp; Cycle 3: Phe+Ser; Cycle 4: Ala; Cycle 5: Gly+Ser; Cycle 6: Asp; Cycle 7: Asp; Cycle 8: Ala; Cycle 9: Pro; with the ADSXS sequence from the CP and AGFAGDDAP-sequence derived from 19-27 actin sequence.

This experiment showed the possibilities of the procedure and also demonstrated that the shift induced by the release of the NH₂-terminal part of CP was sufficiently large to be useful in this invention.

1.6 Identification of the Target Site of a Compound on a Specific Protein Present in a Highly Complex Mixture Such as a Cell Lysate.

Jurkat cells were lysed by incubation with 0.7% CHAPS, 0.5 mM EDTA, 100 mM NaCl, 50 mM Hcpcs, pH7.5 and a protease inhibitor mix. This extract contained 2 mg of total protein/ml.

Five hundred μl were desalted on a MAP5 disposable column equilibrated with 25 mM Tris-HCl pH 8.5, 1 mM EDTA. To one ml of the desalted protein mixture (1 mg), we added 50 μl of acetonitrile and 1.5 μg of endo Lys C. The digest was carried out for 5 h at 37° C.

Five hundred μl of this digest was mixed with 30 μl of the actin-CP endolysine C digest generated in the previous experiment (example 1.5) and 200 μl of 1% TFA in water was added. This mixture was centrifuged and loaded on a 4.6 mm×250 mm. RP-column (Vydac Separations Group). Peptides were eluted exactly as described in FIG. 1A. After 10 min., fractions of 2 min. (2 ml volume) were collected during an additional 50 min. In order to reduce the number of secondary runs, we pooled the fractions as indicated in Table 1.

Each of the combined fractions (A-E) according to Table 1 was vacuum dried and digested with factor Xa. This specific cleavage was carried out in 2.5 ml of buffer containing 40 mM Tris-HCl pH7.3, 60 mM NaCl and 0.12 Units of factor Xa protease. After 2 h, 100 μl of 1% TFA was added and the mixture loaded on the same chromatographic system as in FIG. 1A.

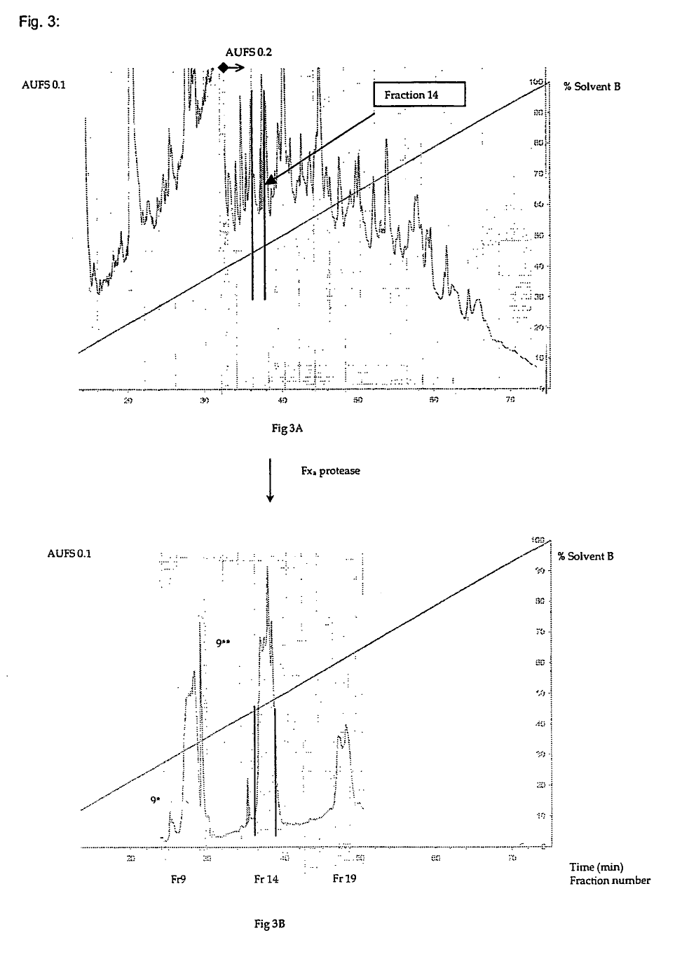

Peptide elution was as in FIG. 1A. The peptide elution profile of pooled fraction D, containing the primary fractions 4-9-14-19-24 is shown in FIG. 3A. We observe peaks emerging from the intervals 9 and 14. Peak 9* eluting in front of interval 9 could not be identified as a peptide. Peak 9**, eluting on the tailing side of interval 9 is derived from the excess of CP which did not react with actin. It is the NH₂-terminal part of CP with sequence Ac-Phe-Ile-Glu-Glu-Arg. This was confirmed by mass spectrometry.

From interval 14 there is a new peak emerging in front of the bulk of unmodified peptides (shown in black). This peak was identified as the cross-linked peptide 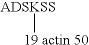
by mass spectrometry and Edman degradation (see also FIG. 2). No other fractions showed peptides that shifted during the secondary run.

This experiment demonstrates that it is possible to specifically select regions, segments or short sequences from proteins which are covalently targeted to compounds that interact with proteins via said regions, segments or short sequences.

TABLE 1
Twenty-five fractions that were collected in the first chromatographic
separation, were collected in five pools (A-E) each containing
the following combinations of primary fractions:

| Pooled fraction N° | Fraction numbers of primary run |
|---|---|
| A | 1 - 6 - 11 - 16 - 21 |
| B | 2 - 7 - 12 - 17 - 22 |
| C | 3 - 8 - 13 - 18 - 23 |
| D | 4 - 9 - 14 - 19 - 24 |
| E | 5 - 10 - 15 - 20 - 25 |

2. Differential Labeling of the Compound-Protein Complexes

2.1, the penicilloyl-moiety could carry one deuterium, more preferably two deuterium, more preferably three deuterium, more preferably four deuterium, preferably more than four deuterium atoms, replacing the corresponding H-atoms in the “light” compound. It should be made clear here that while it seems better to generate large mass differences between the “light” and “heavy” species, for more accurate relative quantification, it is also clear that conversely co-elution or co-migration of the “light” and “heavy” forms of the tagged peptide in the used chromatographic system is less probable with increasing mass difference. Thus the final used mass difference to discriminate between “light” and “heavy” compounds, should be a balance between the largest mass difference for accurate relative quantification and differences still giving rise to identical or very similar chromatographic properties.

2.2, one or more of the amino acids specifying the caspase-1 inhibitory activity could be substituted by an equivalent deuterated amino acid. For instance, the Valine residue could be substituted for d₇-Valine or d₈-Valine. Alternatively, the Alanine residue could be substituted by d₃-Alanine. Thus in sample one, the “light” compound will be linked to its biological targets), while in sample two, the same compound, but now with one or more amino acid(s) substituted by their deuterated homologs will be linked to the biological targets. The peptide mixtures, including the compound-target peptides of samples one and two are mixed. The tagged peptides co-elute and are co-transferred to the mass spectrometer in which they segregate due to their mass differences. The ion intensities, corresponding to both masses are used to calculate the ratios of both tagged peptides, thus of both protein levels or/and activity levels.

2.3, differential labeling will be most conveniently at the phenyl group, present in the sulfenylbenzoyl tag of FSBA. This group can harbour four deuterium atoms. Similar to what is described in the previous examples, protein mixture 1 will be labeled with the H⁴-FSBA reagent (light reagent) while protein mixture 2 will be labeled with FS4 dBA (heavy reagent). After sorting, both of the light and heavy tagged peptides can be compared based on the relative intensities of their respective ions after separation by mass spectrometry.

1. A method to isolate at least one target molecule of a compound comprising a functional group that can be specifically altered, said method comprises the following steps:
(a) adding said compound to a complex mixture of molecules wherein said compound stably interacts with at least one molecule forming a compound-target complex,
(b) separating the resulting complex mixture of molecules and compound-target complexes into fractions via chromatography,
(c) chemically, or enzymatically, or chemically and enzymatically altering said compound present on at least one compound-target complex in each fraction, and
(d) isolating at least one target molecule that interacts with said compound via chromatography, wherein the chromatography of steps (b) and (d) is performed with the same type of chromatography.

2. The method of claim 1, wherein the chromatographic conditions of steps (b) and (d) are the same or substantially similar.

3. A method according to claims 1 wherein said complex mixture of molecules is a complex mixture of proteins.

4. A method according to claim 3 further comprising the cleavage of said complex protein mixture into a protein peptide mixture before performing step (b).

5. A method according to claims 1 wherein said complex mixture of molecules is a protein peptide mixture.

6. The method of claim 1, further comprising the step of identifying the targets.

7. The method of claim 6, wherein said target molecules are proteins or peptides and wherein said identifying step is performed by a method selected from the group consisting of: a tandem mass spectrometric method, Post-Source Decay analysis, measurement of the mass of the peptides, in combination with database searching.

8. The method of claim 7, wherein said identifying step based on the mass measuring of the target peptides is further based on one or more of the following: (a) the determination of the number of free amino groups in the target peptides; (c) the knowledge about the cleavage specificity of the protease used to generate the protein peptide mixture; and (d) the grand average of the hydropathicity of the target peptides.

9. A method to determine the relative amount of the level and/or activity of at least one target protein in more than one sample comprising proteins, comprising the steps of: (a) the addition of a compound comprising a first isotope to a first sample comprising peptides wherein said compound stably interacts with at least one peptide forming a compound-peptide complex; (b) the addition of a compound comprising a second isotope to a second sample comprising peptides wherein said compound stably interacts with at least one peptide forming a compound-peptide complex; (c) combining the protein peptide mixture of the first sample with the protein peptide mixture of the second sample; (d) separating the combined protein peptide mixtures into fractions of peptides via chromatography; (e) chemically, or enzymatically, or chemically and enzymatically, altering said compound present on at least one compound-peptide complex in each fraction; (f) isolating the altered compound-peptide complexes out of each fraction via chromatography, wherein the chromatography is performed with the same type of chromatography as in step (d); (g) performing mass spectrometric analysis of the isolated altered compound-peptide complexes; (h) calculating the relative amounts of the altered compound-peptide complexes in each sample by comparing the peak heights of the identical but differentially, isotopically labelled altered compound-peptide complexes, and (i) determining the identity of said peptides in the altered compound-peptide complexes and their corresponding proteins.

10. The method according to claim 9 wherein the chromatographic conditions of steps (d) and (f) are the same or substantially similar.

11. The method of claim 9 or 10 wherein the determining of the identity of the altered compound-peptide complexes is performed by a method selected from the group consisting of: the tandem mass spectrometric method, Post-Source Decay analysis, measurement of the mass of the peptides, in combination with database searching.

12. The method of claim 11, wherein the determining of the identity of the altered compound-peptide complexes is further based on one or more of the following: (a) the determination of the number of free amino groups in the peptides; (c) the knowledge about the cleavage specificity of the protease used to generate the protein peptide mixture; and (d) the grand average of the hydropathicity of the peptides.


SEQUENCE LISTING (8 sequences)

SEQ ID NO: 1 - protein, 5 aa, Artificial Sequence
  purely synthetic peptide (used in figure 3B and is acetylated).
FIEGR

SEQ ID NO: 2 - protein, 11 aa, Artificial Sequence
  Purely synthetic peptide (The peptide used in example 1.2 has an acetylated free alpha-NH2-terminus and a free COOH-terminus).
FIEGRADSKSS

SEQ ID NO: 3 - protein, 10 aa, Artificial Sequence
  purely synthetic peptide (The peptide in example 1.3; the Asp gro up carries an aldehyde, the first Ala is acetylated).
AAIEGRYVAD

SEQ ID NO: 4 - protein, 4 aa, Artificial Sequence
  Purely synthetic peptide (The peptide in example 1.3; the Asp gro up carries an aldehyde, the Tyr is acetylated).
YVAD

SEQ ID NO: 5 - protein, 6 aa, Artificial Sequence
  Purely synthetic peptide (The peptide used in example 1.5 and Lys carries 19-actin-50).
ADSKSS

SEQ ID NO: 6 - protein, 5 aa, Artificial Sequence
  Purely synthetic peptide (Peptide used in example 1.5; sequence from the compound peptide).
ADSXS

SEQ ID NO: 7 - protein, 9 aa, Artificial Sequence
  Purely synthetic peptide (Peptide used in example 1.5; sequence derived from 19-27 actin sequence).
AGFAGDDAP

SEQ ID NO: 8 - protein, 5 aa, Artificial Sequence
  Purely synthetic peptide (The peptide is used in example 1.6 the NH2-terminal part of the compound peptide; the Phe is acetylated).
FIEER